(19) United States
(12) Patent Application Publication
(10) Pub. No.: US 2024/0285950 A1
(43) Pub. Date: Aug. 29, 2024

(54) METHOD AND APPARATUS FOR CLOSED-LOOP NEUROSTIMULATION WITH PATIENT CONTROL

(71) Applicant: Boston Scientific Neuromodulation Corporation, Valencia, CA (US)

(72) Inventors: Tianhe Zhang, Studio City, CA (US); Andrew James Haddock, Los Angeles, CA (US)

(21) Appl. No.: 18/440,468

(22) Filed: Feb. 13, 2024

Related U.S. Application Data
(60) Provisional application No. 63/448,008, filed on Feb. 24, 2023.

(51) Int. Cl. A61N 1/36; G16H 10/20; G16H 40/67

(52) U.S. Cl. CPC A61N 1/36132; A61N 1/36125; G16H 10/20; G16H 40/67

(57) ABSTRACT

An implantable stimulator can control delivery of the neurostimulation to a patient according to a stimulation configuration and adjust the stimulation configuration by using a closed-loop control algorithm. In an example, the system may include a remote controller (RC) configured for use by the patient. The RC may transmit stimulator adjustment information to the implantable stimulator. The stimulator adjustment information may include direct control adjustment information for adjusting the stimulation configuration and adaptation adjustment information for adjusting the closed-loop control algorithm. The RC may generate the stimulator adjustment information based on patient adjustment instructions. The RC includes a user interface configured to receive a patient input and to determine the patient adjustment instructions by interpreting the patient input according to an RC configuration that is programmable for modifying capabilities of the RC in adjusting the stimulation configuration and adjusting the closed-loop control algorithm.

### CLAIM OF PRIORITY

This application claims the benefit of U.S. Provisional Application No. 63/448,008 filed on Feb. 24, 2023, which is hereby incorporated by reference in its entirety.

### TECHNICAL FIELD

This document relates generally to neurostimulation and more particularly to a neurostimulation system with closed-loop control of stimulation delivery that uses patent input to adjust the closed-loop control.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Stimulation parameters specifying the spatial aspects may determine where to place electrodes and/or which electrodes to select for delivering the neurostimulation pulses. In a neurostimulation system with closed-loop control, one or more responses of the patient to the delivery of the neurostimulation pulses may be monitored to adjust the stimulation parameters to maintain intended therapeutic effectiveness while avoiding or reducing undesirable effects.

### SUMMARY

An Example (e.g., “Example 1”) of a system for delivering neurostimulation to a patient and controlling the neurostimulation by a user is provided. The neurostimulation is to be delivered from an implantable stimulator configured to control the delivery of the neurostimulation according to a stimulation configuration and to adjust the stimulation configuration by using a closed-loop control algorithm. The system may include a remote controller (RC) configured for use by the patient. The RC may include an RC telemetry circuit, an RC programming control circuit, and an RC user interface. The RC telemetry circuit may be configured to provide wireless communication between the RC and the implantable stimulator and to transmit stimulator adjustment information to the implantable stimulator. The stimulator adjustment information may include direct control adjustment information for adjusting the stimulation configuration and adaptation adjustment information for adjusting the closed-loop control algorithm. The RC programming control circuit may be configured to generate the stimulator adjustment information based on patient adjustment instructions. The RC user interface may include an RC user input device and a stimulator programming circuit. The RC user input may be configured to receive a patient input. The stimulator programming circuit may be configured to determine the patient adjustment instructions by interpreting the patient input according to an RC configuration that is programmable for modifying capabilities of the RC in adjusting the stimulation configuration and adjusting the closed-loop control algorithm.
In Example 2, the subject matter of Example 1 may optionally be configured such that the stimulator programming circuit is configured to enable and disable each function of RC functions using the user input according to the RC configuration. The RC functions allow the delivery of the neurostimulation from the implantable stimulator to be adjusted using the RC and including one or more functions each allowing one or more aspects of the closed-loop control algorithm to be adjusted using the RC.
In Example 3, the subject matter of any one or any combination of Examples 1 and 2 may optionally be configured to include the implantable stimulator, which includes a stimulation output circuit configured to deliver the neurostimulation, a sensing circuit configured to sense a signal indicative of a response of the patient to the delivery of the neurostimulation, and a stimulation control circuit configured to control the delivery of the neurostimulation using the stimulation configuration and to adjust the stimulation configuration by using the closed-loop control algorithm and the sensed signal and an input to the closed-loop control algorithm.
In Example 4, the subject matter of any one or any combination of Examples 1 to 3 may optionally be configured such that the RC is configured to track an RC usage indicative of the transmission of the stimulator adjustment information to the implantable stimulator.
In Example 5, the subject matter of any one or any combination of Examples 1 to 4 may optionally be configured to further include an RC programming circuit configured to receive RC programming information and to determine the RC configuration based on the RC programming information.
In Example 6, the subject matter of Example 5 may optionally be configured such that the RC user interface includes the RC programming circuit, and the RC user input device is configured to receive the RC programming information.
In Example 7, the subject matter of Example 5 may optionally be configured such that the RC is configured to receive the RC programming information using the RC telemetry circuit and to program the RC configuration using the received RC programming information.
In Example 8, the subject matter of Example 7 may optionally be configured to further include a clinician's programmer (CP). The CP includes a CP telemetry circuit, a CP programming control circuit, and a CP user interface. The CP telemetry circuit is configured to provide wireless communication between the CP and the implantable stimulator and wireless communication between the CP and the RC and to transmit the RC programming information to the RC. The CP programming control circuit is configured and to generate the RC programming information based on the RC configuration. The CP user interface includes the RC programming circuit and a CP user input device configured to receive a user input including the RC programming information.
In Example 9, the subject matter of any one or any combination of Examples 7 and 8 may optionally be configured such that the CP user interface includes a stimulator programming circuit configured to determine the stimulation configuration and the closed-loop control algorithm based on the user input, the CP programming control circuit is configured to generate stimulator programming information based on the stimulation configuration, and the CP telemetry circuit is further configured to transmit the stimulator programming information to the implantable stimulator. The stimulator programming information includes information for adjusting the stimulation configuration and information for adjusting the closed-loop control algorithm
In Example 10, the subject matter of any one or any combination of Examples 5 to 9 may optionally be configured such that the RC programming circuit is configured to allow the RC configuration to be defined to enable the RC user input device to receive the patient input for at least one of adjusting the stimulation configuration or adjusting the closed-loop control algorithm.
In Example 11, the subject matter of Example 10 may optionally be configured such that the RC programming circuit is configured to allow the RC configuration to be defined to allow the RC user input device to receive the patient input for adjusting the stimulation configuration while overriding the closed-loop control algorithm.
In Example 12, the subject matter of any one or any combination of Examples 10 and 11 may optionally be configured such that the RC programming circuit is configured to allow the RC configuration to be defined to enable the RC user input device to receive the patient input for causing gradual shifts in at least one of a setpoint or a tolerance range around that setpoint, the setpoint being a parameter of the closed-loop control algorithm associated with a measurable result of the delivery of the neurostimulation.
In Example 13, the subject matter of any one or any combination of Examples 10 to 12 may optionally be configured such that the RC programming circuit is configured to allow the RC configuration to be defined to enable the RC user input device to receive the patient input as one or more metrics, and the stimulator programming circuit is configured to adjust the stimulation configuration directly using the one or more metrics.
In Example 14, the subject matter of Example 13 may optionally be configured such that the RC programming circuit is configured to allow the RC configuration to be defined to enable the RC user input device to receive at least one of a weighting factor or a scaling factor for each parameter of the one or more metrics, and the stimulator programming circuit is configured to adjust the stimulation configuration directly using the each parameter and the respective at least one of the weighting factor or the scaling factor.
In Example 15, the subject matter of any one or any combination of Examples 10 to 14 may optionally be configured such that the RC programming circuit is configured to allow the RC configuration to be defined to enable the RC user input device to receive the patient input as a response to a survey, and the stimulator programming circuit is configured to adjust at least one of the stimulation configuration or the closed-loop control algorithm based on the response to the survey.
An example (e.g., “Example 16”) of a method for delivering neurostimulation to a patient and controlling the neurostimulation by a user is also provided. The neurostimulation is delivered from an implantable stimulator configured to control the delivery of the neurostimulation according to a stimulation configuration and to adjust the stimulation configuration by using a closed-loop control algorithm. The method may include providing a remote controller (RC) configured to be used by the patient and to wirelessly communicate with the implantable stimulator, receiving a patient input using the RC, determining patient adjustment instructions by interpreting the patient input according to an RC configuration that is programmable for modifying capabilities of the RC in adjusting the stimulation configuration and adjusting the closed-loop control algorithm, generating stimulator adjustment information based on the patient adjustment instructions, and transmitting the stimulator adjustment information from the RC to the implantable stimulator. The stimulator adjustment information may include direct control adjustment information for adjusting the stimulation configuration directly and adaptation adjustment information for adjusting the closed-loop control algorithm
In Example 17, the subject matter of Example 16 may optionally further include tracking a usage of the RC in generating the direct control adjustment information and a usage of the RC in generating the adaptation adjustment information.
In Example 18, the subject matter of any one or any combination of Examples 16 and 17 may optionally further include receiving RC user input using a user interface of the RC and adjusting the RC configuration using the RC user input.
In Example 19, the subject matter of any one or any combination of Examples 16 to 18 may optionally further include receiving RC programming information using a telemetry circuit of the RC and adjusting the RC configuration using the RC programming information.
In Example 20, the subject matter of Example 19 may optionally further include providing a clinician's programmer (CP) configured for use by the user, receiving a CP user input using a user interface of the CP, and generating the RC programming information based on the CP user input.
In Example 21, the subject matter of any one or any combination of Examples 16 to 20 may optionally further include adjusting the RC configuration to enable the RC to receive the patient input for at least one of adjusting the stimulation configuration or adjusting the closed-loop control algorithm.
In Example 22, the subject matter of any one or any combination of Examples 16 to 21 may optionally further include adjusting the RC configuration to enable the RC to receive the patient input for causing gradual shifts in at least one of a setpoint or a tolerance range around that setpoint, the setpoint being a parameter of the closed-loop control algorithm associated with a measurable result of the delivery of the neurostimulation.
In Example 23, the subject matter of any one or any combination of Examples 16 to 22 may optionally further include adjusting the RC configuration to enable the RC to receive the patient input as one or more metrics that are used to directly adjust the stimulation configuration.
In Example 24, the subject matter of any one or any combination of Examples 16 to 23 may optionally further include adjusting the RC configuration to enable the RC to receive the patient input as a response to a survey, the response used to adjust at least one of the stimulation configuration or the closed-loop control algorithm.
An example (e.g., “Example 25”) of a non-transitory computer-readable storage medium is also provided. The non-transitory computer-readable storage medium includes instructions, which when executed by a system, cause the system to perform a method for delivering neurostimulation to a patient and controlling the neurostimulation by a user. The neurostimulation is delivered from an implantable stimulator configured to control the delivery of the neurostimulation according to a stimulation configuration and to adjust the stimulation configuration by using a closed-loop control algorithm The method may include communicating with the implantable stimulator wirelessly using a remote controller (RC) configured for use by the patient, receiving a patient input using the RC, determining patient adjustment instructions by interpreting the patient input according to an RC configuration that is programmable for modifying capabilities of the RC in adjusting the stimulation configuration and adjusting the closed-loop control algorithm, generating stimulator adjustment information based on the patient adjustment instructions, and transmitting the stimulator adjustment information from the RC to the implantable stimulator. The stimulator adjustment information includes direct control adjustment information for adjusting the stimulation configuration directly and adaptation adjustment information for adjusting the closed-loop control algorithm.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a neurostimulation system allowing a patient to adjust closed-loop control of neurostimulation, such as may be implemented in the neurostimulation system of FIG. 1, 5, or 6.

FIG. 10 illustrates an embodiment of a remote controller (RC), such as may be implemented in the neurostimulation system of FIG. 9.

FIG. 11 illustrates an embodiment of a clinician's programmer (CP), such as may be implemented in the neurostimulation system of FIG. 9.

FIG. 12 illustrates an embodiment of portions of a display on a user interface allowing for programming of an RC configuration for interpretation of patient input.

FIG. 13 illustrates an embodiment of portions of a display on a user interface allowing for adjustment of a setpoint of the closed-loop control.

FIG. 14 illustrates an embodiment of portions of a display on a user interface allowing for adjustment of a therapeutic window of the closed-loop control.

FIG. 15 illustrates an embodiment of portions of a display on a user interface allowing for use of patient input as metric(s) for adjusting stimulation parameter(s).

FIG. 16 illustrates an embodiment of portions of a display on a user interface allowing for use of patient input as metric(s) for adjusting stimulation parameter(s) with step scaling.

FIG. 17 illustrates an embodiment of portions of a display on a user interface allowing for use of patient input as metric(s) for adjusting stimulation parameter(s) with weight scaling.

FIG. 18 illustrates an embodiment of portions of a display on a user interface showing a warning message on accepting patient input.

FIGS. 19A-19B illustrate an embodiment of portions of a display on a user interface (FIG. 19A) and a decision tree (FIG. 19B) used in survey-based adjustments of the closed-loop control of neurostimulation.

FIG. 20 illustrates an embodiment of a method for adjusting closed-loop control of neurostimulation based on patient input.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that includes closed-loop control of delivery of stimulation energy to a patient and allows the patient to adjust the closed-loop control to an extent that can be programmable. In various embodiments, the neuromodulation system can include an implantable device configured to deliver neurostimulation (also referred to as neuromodulation) therapies, such as deep brain stimulation (DBS), spinal cord stimulation (SCS), peripheral nerve stimulation (PNS), and vagus nerve stimulation (VNS), and one or more external devices configured to program and/or adjust the implantable device and to monitor the conditions of the patient and performance of the implantable device. An external patient device can be configured for use by the patient, including controlling the delivery of the neurostimulation to a limited extent. An external clinician device can be configured for use by a user such as a healthcare professional trained for programming the neurostimulation system, including setting the limited extent to which the delivery of the neurostimulation can be controlled using the external patient device.

A neurostimulation system with closed-loop control of delivery of stimulation to the patient can operate passively to maintain a setpoint using an internally acquired signal (e.g., a neural or other physiological signal sensed from the patient), with little or no patient intervention, beyond an initial calibration. The setpoint can be a physiological parameter that is measured from a signal sensed from the patient and used to control the delivery of the stimulation to maintain its value within a target range. Manual intervention, although more associated with an open-loop system, can serve as an additional variable that either supplements an existing closed-loop control algorithm or, under some circumstances, operates as an alternative algorithm when needed (e.g., when the closed-loop control algorithm stops providing the intended effect).

The present subject matter provides systems and methods for adjusting (e.g., modifying or tuning) a closed-loop control algorithm using patient input (e.g., interaction or feedback). It is noted that the patient input is used to adjust the algorithm itself, while patient input of the same or different type can also be used as an input signal to the algorithm. In this document, unless noted otherwise, a “patient” includes a person receiving treatment delivered from, and/or monitored using, a neurostimulation system according to the present subject matter. A “user” includes a person who can program the neurostimulation system including its various components and can be a physician, other caregiver who examines and/or treats the patient using the neurostimulation system, or other person who participates in the examination and/or treatment of the patient using the neurostimulation system (e.g., a technically trained representative, a field clinical engineer, a clinical researcher, or a field specialist from the manufacturer of the neurostimulation system). A “patient input” includes a command influencing the operation of the neurostimulation system that is entered by the patient (or a user on behave of the patient), for example by using the external patient device. A “user input” includes a command influencing the operation of the neurostimulation system that is entered by the user, for example by using the external clinician device. The patient input can include information for adjusting the closed-loop control algorithm. The user input can include information defining a scope of participation of the patient in adjusting the closed-loop control algorithm.

In various embodiments, features of patient participation in the adjustment of the closed-loop control algorithm can be programmed into the neurostimulation system to be enabled by a user and/or by the patient, depending on the type or extent of the patient participation intended by the user. For example, in the neurostimulation system can including the implantable device, the external patient device, and the external clinician device, the external patient device can be configured to allow one or more aspects of the closed-loop control algorithm to be adjusted by the patient, and the external clinician device can be configured for the user to enable the one or more aspects of the closed-loop control algorithm for adjustment using the external patient device. The external patient device can also be configured to monitor the amount of patient participation in adjusting the closed-loop control algorithm.

Examples of features of patient participation in adjustment of closed-loop control of neurostimulation according to the present subject matter include:
A. Toggling. The patient can choose between overriding and adjusting the instant closed-loop control. For example, overriding may be desired when the patient feels very uncomfortable and believes that the neurostimulation is causing the discomfortable, and adjustment may be desired when the patient has learned that certain adjustments may improve the effectiveness of the neurostimulation.
B. Setpoint. The patient can adjust a reference (e.g., a setpoint for stimulation) in the closed-loop control, for example by pushing a button before starting a physical activity or changing a body posture.
C. Weighting. The patient can adjust one or more feedback variables and/or scaling or weighting of one or more feedback variables in the closed-loop control, for example to obtain a desirable effect or degree of effect,
D. Survey. The closed-loop control can be adjusted using subjective information from the patient, for example using a priori survey.
   In various embodiments, the neurostimulation system can be configured to support any one or any combination of these examples and any other features of patient participation in adjustment of closed-loop control of neurostimulation.

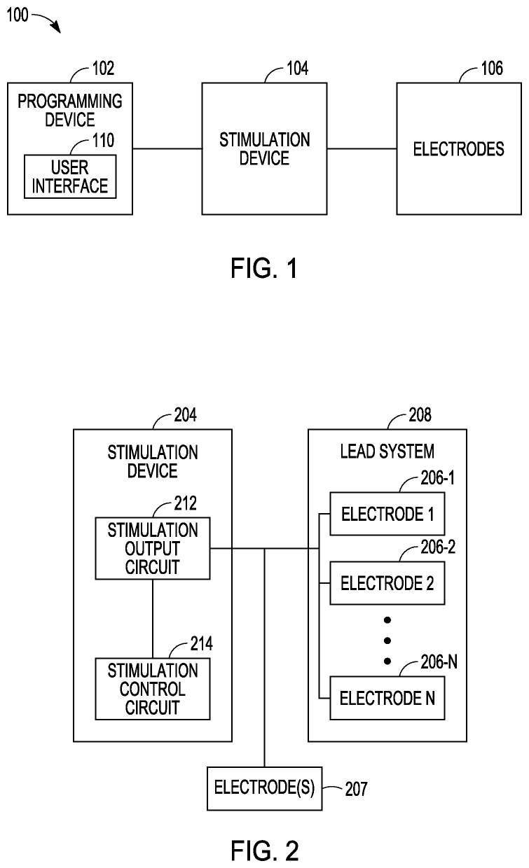

FIG. 1 illustrates an embodiment of a neurostimulation system 100. System 100 includes electrodes (also referred to as contacts) 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for spinal cord stimulation (SCS) applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 (also referred to as an electrode array in this document) distributed in the one or more leads. The plurality of electrodes 206 includes electrode 206-1, electrode 206-2, . . . electrode 206-N, each being a single electrically conductive contact providing for an electrical interface between stimulation output circuit 212 and tissue of the patient (and therefore also referred to as a contact in this document), where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

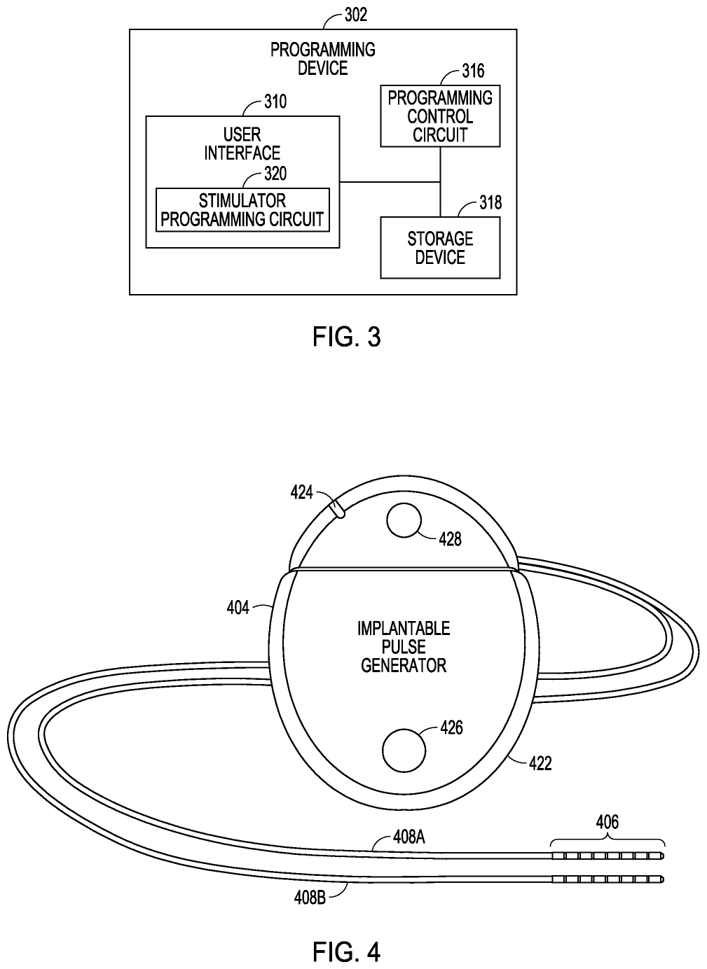

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulator programming circuit 320. Storage device 318 stores information used by programming control circuit 316 and stimulator programming circuit 320, such as information about a stimulation device that relates the neurostimulation program to the plurality of stimulation parameters. In various embodiments, stimulator programming circuit 320 can be configured to support one or more functions allowing for programming of stimulation devices, such as stimulation device 104 including its various embodiments as discussed in this document, according to one or more selected neurostimulation programs and/or one or more closed-loop control algorithms.

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” can include the pattern of neurostimulation pulses including the one or more stimulation fields, or at least various aspects or parameters of the pattern of neurostimulation pulses including the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

In various embodiments, circuits of neurostimulation system 100, including its various embodiments discussed in this document, may be implemented using a combination of hardware and software. For example, the circuit of user interface 110, stimulation control circuit 214, programming control circuit 316, and stimulator programming circuit 320, including their various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrodes (also referred to as contacts) 406 for contacting a tissue site targeted for electrical neurostimulation. As illustrated in FIG. 4, leads 408A and 408B each include 8 electrodes 406 at the distal end. The number and arrangement of leads 408A and 408B and electrodes 406 as shown in FIG. 4 are only an example, and other numbers and arrangements are possible. In various embodiments, the electrodes are ring electrodes. In various embodiments applying DBS or SCS, the implantable leads and electrodes may be configured by shape and size to provide electrical neurostimulation energy to a neuronal target included in the patient's brain or configured to provide electrical neurostimulation energy to target nerve cells in the patient's spinal cord.

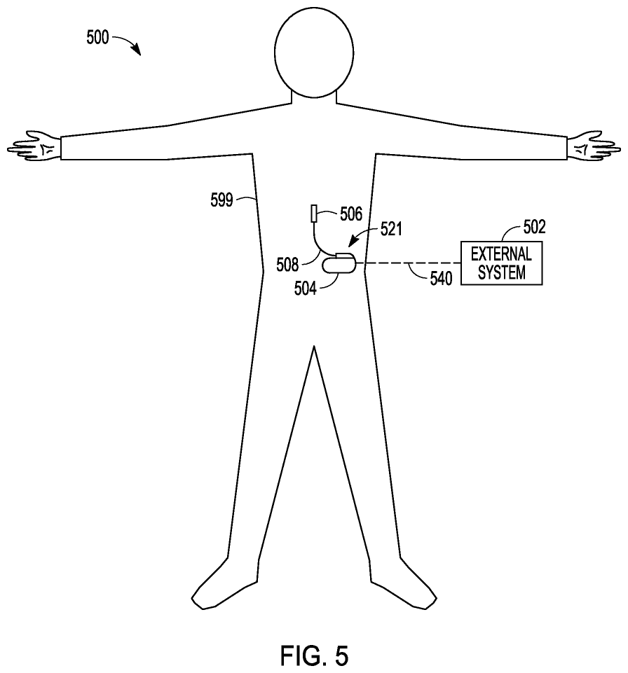

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes (also referred to as contacts) 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and shapes of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

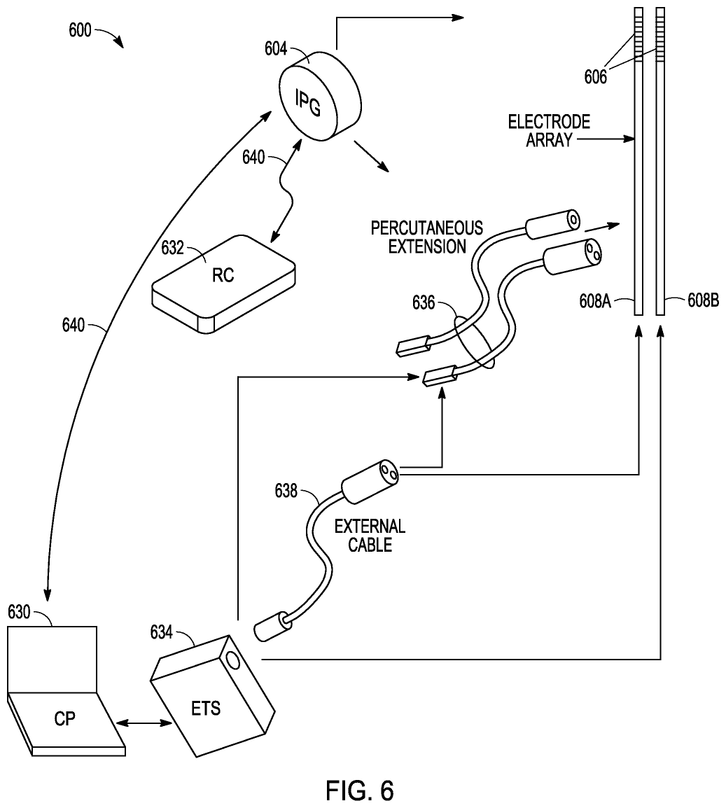

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS, also referred to as external trial modulator, ETM) 634. IPG 604 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with IPG 604 representing an embodiment of stimulation device 104, electrodes (also referred to as contacts) 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 640. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632. In various embodiments, RC 632 can be a dedicated device or a general-purpose device configured to perform the functions of RC 632, such as a smartphone, a tablet computer, or other smart/mobile device.

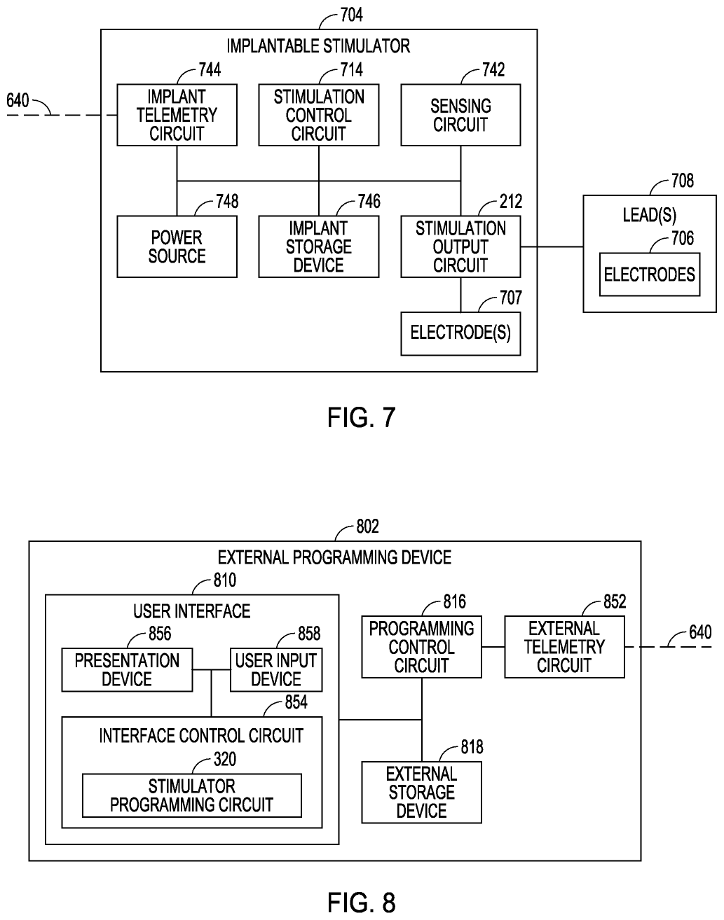

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes (also referred to as contacts) 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more ESG signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 (e.g., by installing a new application in a smart device such as a smartphone) and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS or SCS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time (and allows the user to do so) to shape the stimulation like a piece of clay (pushing/pulling one spot at a time).

Programming control circuit 816 represents an example of programming control circuit 316 and generates the plurality of stimulation parameters, which is to be transmitted to implantable stimulator 704, based on a specified neurostimulation program (e.g., the pattern of neurostimulation pulses as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes stimulator programming circuit 320.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

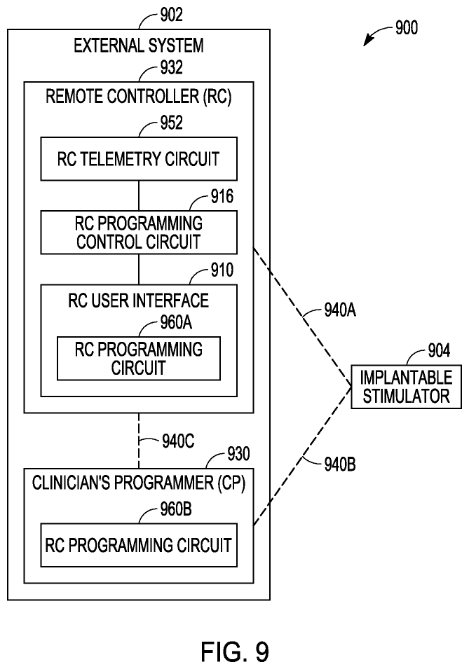

FIG. 9 illustrates an embodiment of a neurostimulation system 900, which allows a patient to adjust closed-loop control of neurostimulation. System 900 includes an implantable stimulator 904 and an external system 902 that includes a remote controller (RC) 932 and a clinician's programmer (CP) 930. System 900 can be implemented in a neurostimulation system such as systems 100, 500, or 600. In various embodiments, implantable stimulator 904 can be implemented in an implantable medical device, such as IPG 404, IPG or implantable stimulator 504, IPG 604, or implantable stimulator 704. External system 902 can be implemented in an external system including one or more programming devices, such as external system 502, CP 630 and RC 632, or external programming device 802. For example, when system 900 is implemented in system 600, implantable stimulator 904 is implemented in IPG 604, RC 932 is implemented in RC 632, and CP 930 is implemented in CP 630. In various embodiments, external system 902 can be implemented as a single device or as two or more devices.

Implantable stimulator 904 can deliver the neurostimulation, control the delivery of the neurostimulation using a stimulation configuration, and adjust the stimulation configuration by using a closed-loop control algorithm. The stimulation configuration includes stimulation parameters defining one or more stimulation waveforms and one or more stimulation fields. Implantable stimulator 904 can receive an input to the closed-loop control algorithm and to adjust one or more stimulation parameters of the stimulation configuration using the received input according to the closed-loop control algorithm. For example, implantable stimulator 904 can sense a signal indicative of a response of the patient to the delivery of the neurostimulation, measure a parameter from the sensed signal, and use the parameter as the input to the closed-loop control algorithm. When implantable stimulator 904 is implemented in implantable stimulator 904, stimulation output circuit 212 can be configured to deliver the neurostimulation, stimulation control circuit 714 can be configured to control the delivery of the neurostimulation using the stimulation configuration and to adjust the stimulation configuration by using the closed-loop control algorithm, and sensing circuit 742 can be configured to sense the signal indicative of the response of the patient to the delivery of the neurostimulation.

RC 932 can be configured for use by the patient and can communicate with implantable stimulator 904 via a wireless communication link 940A. CP 930 can be configured for use by a user and can communicate with implantable stimulator 904 via a wireless communication link 940B. RC 932 and CP 930 can communicate with each other via a wireless communication link 940C. Wireless communication links 940A-C can each be an example of wireless communication link 640.

RC 932 can include an RC telemetry circuit 952, an RC programming control circuit 916, and an RC user interface 910. RC telemetry circuit 952 can represent an example of external telemetry circuit 852 and can provide wireless communication between RC 932 and implantable stimulator 904. RC telemetry circuit 952 can transmit stimulator adjustment information to implantable stimulator 904. The stimulator adjustment information can include direct control adjustment information for adjusting the stimulation configuration and/or adaptation adjustment information for adjusting the closed-loop control algorithm. RC programming control circuit 916 can represent an example of programming control circuit 816 and can generate the stimulator adjustment information based on patient adjustment instructions. RC user interface 910 can represent an example of user interface 810 and can receive a patient input and determine the patient adjustment instructions by interpreting the patient input according to an RC configuration. The RC configuration is programmable for modifying capabilities of RC 932 in adjusting the stimulation configuration and adjusting the closed-loop control algorithm.

In the illustrated embodiment, RC 932 includes an RC programming circuit 960A, and CP 930 includes an RC programming circuit 960B. RC programming circuits 960A-B each represent an RC programming circuit that can receive RC programming information and determine the RC configuration based on the RC programming information (i.e., programming the RC configuration into RC 632 based on the RC programming information). In various embodiments, either or both of RC 932 and CP 630 can include the RC programming circuit. RC programming circuit 960A can receive the RC programming information from the user using the RC user interface 910, which can provide only authorized users with access to the programming of the RC configuration. Alternatively, or additionally, RC programming circuit 960A can receive the RC programming information from CP 630, which can receive the RC programming information from the user using its user interface.

In various embodiment, the closed-loop control algorithm can reside on implantable stimulator 904 and/or RC 932. In one example, the closed-loop control algorithm resides on implantable stimulator 904, such that implantable stimulator 904 can execute the closed-loop control algorithm without communicating to RC 932 unless a change to the closed-loop control algorithm or another device setting is to be made. In another example, the closed-loop control algorithm resides on RC 932. This may increase capability and/or flexibility of the closed-loop control algorithm while requiring increased capability of RC 932 and reliability of its communications with implantable stimulator 904.

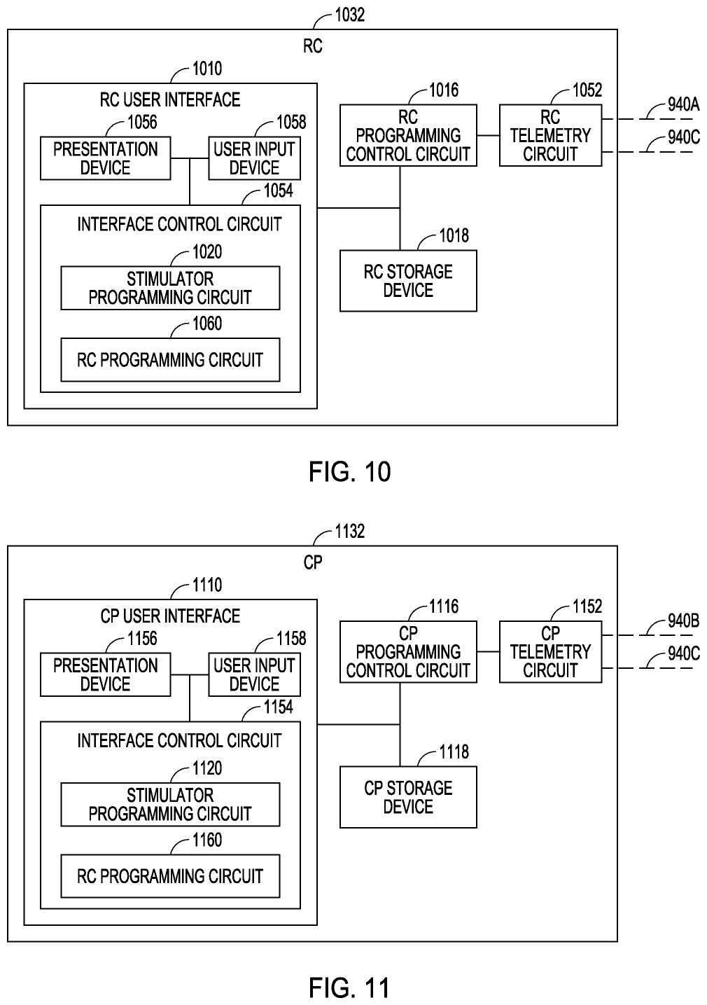

FIG. 10 illustrates an embodiment of an RC 1032, which can represent an example of RC 932 and an example of external programming device 802 configured to be an RC. RC 1032 includes an RC telemetry circuit 1052, an RC programming control circuit 1016, an RC storage device 1018, and an RC user interface 1010, which can represent an example of external telemetry circuit 852, external storage device 818, programming control circuit 816, and user interface 810, respectively.

RC external telemetry circuit 1052 can provide communication between RC 1032 via wireless communication link 904A and implantable stimulator 904 and communication between RC 1032 and CP 930 via wireless communication link 904C. RC external telemetry circuit 1052 can receive the RC programming information including the RC configuration from CP 930, when the RC configuration is to be programmed using CP9 930, and can transmit RC usage information to CP 930. The RC usage information is recorded by RC 1032 for tracking usage of each function of functions of RC 1032 by the patient (and/or a person using RC 1032 on behalf on the patient). RC external telemetry circuit 1052 can transmit stimulator adjustment information to implantable stimulator 904. The stimulator adjustment information is for adjusting the delivery of the neurostimulation from implantable stimulator 904 based on request entered using RC 1032 by the patient (or the person on behalf on the patient). The stimulator adjustment information includes direct control adjustment information for adjusting the stimulation configuration and/or adaptation adjustment information for adjusting the closed-loop control algorithm.

RC programming control circuit 1016 can generate the stimulator adjustment information based on patient adjustment instructions. Patient adjustment instructions can include direct control instructions for adjusting the stimulation configuration and adaptation instructions for adjusting the closed-loop control algorithm. The direct control instructions are provided to directly cause adjustment of stimulation parameters in implantable stimulator 904. The adaptation instructions are provided to cause adjustment of the closed-loop control algorithm, which can indirectly cause the stimulation parameters to be adjusted.

RC storage device 1018 can store information required for operation of RC 1032. The store information can include instructions to be executed by RC 1032 to perform various methods and functions that can be performed by RC 1032 as discussed in this document.

RC user interface 1010 can include a presentation device 1056, a user input device 1058, and an interface control circuit 1054, which can represent an example of presentation device 856, user input device 858, and interface control circuit 854, respectively. User input device 1058 can receive a patient input and optionally a user input (e.g., the RC programming information, when RC 1032 is used for the user to program the RC configuration). Interface control circuit 1020 includes a stimulator programming circuit 1020, which can represent an example of stimulator programming circuit 320. Stimulator programming circuit 1020 can determine the patient adjustment instructions based on the patient input and the RC configuration by interpreting the patient input according to the RC configuration. Interface control circuit 1020 can optionally also include an RC programming circuit 1060, which can represent an example of RC programming circuit 960A. For example, when RC 1032 is used for the user to program the RC configuration, RC programming circuit 1060 can determine the RC configuration based on the user input including the RC programming information. This allows the user, when authorized, to define the programming capabilities of RC 1032 for being used by the patient.

The RC configuration defines the programming capabilities of RC 1032 and relates the patient input received by RC 1032 to the patient adjustment instructions determined by RC 1032. For example, the RC configuration allows user input device 1058 to be used to enable and disable each of RC functions allowing the delivery of the neurostimulation from implantable stimulator 904 to be adjusted using RC 1032. These RC functions include one or more functions each allowing one or more aspects of the closed-loop control algorithm executed by implantable stimulator 904 to be adjusted using RC 1032.

FIG. 11 illustrates an embodiment of a CP 1130, which can represent an example of CP 930 and an example of external programming device 802 configured to be a CP. CP 1130 includes a CP telemetry circuit 1152, a CP programming control circuit 1116, a CP storage device 1118, and a CP user interface 1110, which can represent an example of external telemetry circuit 852, external storage device 818, programming control circuit 816, and user interface 810, respectively.

CP external telemetry circuit 1152 can provide communication between CP 1130 and implantable stimulator 904 via wireless communication link 940B and communication between CP 1130 and RC 932 via wireless communication link 940C. CP external telemetry circuit 1152 can transmit stimulator programming information to implantable stimulator 904 and can receive acquired information from implantable stimulator 904. The stimulator programming information can include information for adjusting the stimulation configuration and/or information for adjusting the closed-loop control algorithm. The acquired information can include, for example, sensed signals indicative of conditions of the patient and information indicative of performance of implantable stimulator 904. CP external telemetry circuit 1152 can also transmit the RC programming information to RC 932 (when CP 1030 is used for the user to program the RC configuration) and can receive the RC usage information from RC 932.

CP programming control circuit 1118 can generate the stimulator programming information based on the stimulation configuration. When CP 1030 is used for the user to program the RC configuration, CP programming control circuit 1118 can also generate the RC programming information based on the RC configuration.

CP storage device 1118 can store information required for operation of CP 1130. The store information can include instructions to be executed by CP 1130 to perform various methods and functions that can be performed by CP 1130 as discussed in this document.

CP user interface 1110 can include a presentation device 1156, a user input device 1158, and an interface control circuit 1154, which can represent an example of presentation device 856, user input device 858, and interface control circuit 854, respectively. User input device 1158 can receive a user input, which can include the RC programming information when CP 1130 is used for the user to program the RC configuration. Interface control circuit 1120 includes a stimulator programming circuit 1120, which can represent an example of stimulator programming circuit 320. Stimulator programming circuit 1120 can determine the stimulation configuration based on the user input. Interface control circuit 1120 can optionally also include an RC programming circuit 1160, which can represent an example of RC programming circuit 960B. For example, when CP 1130 is used for the user to program the RC configuration, RC programming circuit 1160 can determine the RC configuration based on the user input including the RC programming information. This allows the user to define the programming capabilities of RC 932 (including RC 1032 as an example) for being used by the patient.

In a neurostimulation system (an example of system 900) that includes RC 1032 and CP 1130, at least one of RC 1032 or CP 1130 can be used by the user to programming the RC configuration for RC 1032. Thus, the neurostimulation system includes RC programming circuit 1060 in RC 1032 and/or RC programming circuit 1160 in CP 1130. In various embodiments, the neurostimulation system can be configured to allow the user to programming the RC configuration for RC 1032 by using:
RC 1032 only (with user input device 1058 protected, for example by using a password, from programming the RC configuration by unauthorized users including the patient);
CP 1130 only; and
RC 1032 and CP 1130 (e.g., whichever is available or more convenient at the instant).

The RC configuration can define function of each feature of user input device 1058 of RC 1032. Such features of user input device 1058 can include one or more buttons and one or more arrows. For example, user input device 1058 can include a button and a pair of arrows pointing to the opposite directions. Each “button” or “arrow” can be a physical feature of user input device 1058, for example when RC 1032 is a dedicated device, or can be symbols on a touchscreen (functioning as presentation device 1056 and user input device 1058), for example when RC 1032 is a smartphone installed with an RC application. Examples of functions of such features of user input device 1058 that can be defined by the RC figuration include:
Button Event: A button is clicked to enter one or more types of command as the patient input. The “events” can be counted in number of clicks, in duration (e.g., button held for seconds), in number of clicks received over a specified time window, etc., each representing a specific command.
Direct Control: An arrow is clicked to directly adjust a stimulation parameter in increments (e.g., intensity up or down, with a specified amount of increment per click).
Adaptive (Scale): An arrow is clicked to adjust the closed-loop control algorithm. For example, a rapid succession of clicks, along with direct control, can temporarily increase or decrease the step size, slew rate, or speed and magnitude of other behavior(s) of the control algorithm.
Adaptive (Set): An arrow is clicked to adjust the closed-loop control algorithm by changing a setpoint, a therapeutic window, and/or another preconfigured reference point.

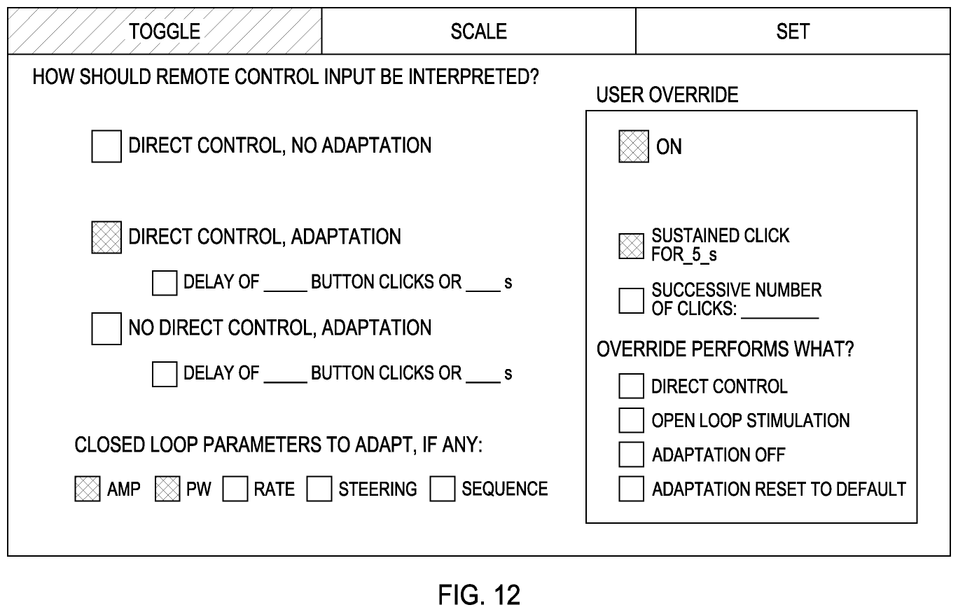

Examples of functions of RC 1032 that can be adjustably defined by the user by programming the RC configuration are discussed below with references to FIGS. 12-19. Each of FIGS. 12-19 shows a portion of a display on presentation device 1056 or 1156 as an example, rather than a restriction, that illustrates a concept of user interface capabilities that does not necessarily reflect a specific design for the organization and layout of the contents to be displayed. In the following discussion (with references to FIGS. 12-19), each function of “the RC programming circuit” can include RC programming circuit 1060 and/or RC programming circuit 1160, depending on whether the RC configuration can be programmed using RC 1032, CP 1130, or both, as discussed above. In the following discussion (with references to FIGS. 12-19), “the RC” refer to a remote controller intended for use by a patient or a person on behalf of the patient, with RC 632, RC 932, and 1032 each being an example.

### Example 1: Toggling

The RC programming circuit can allow the RC configuration to be programmed to enable the user input device of the RC to receive the patient input for producing the direct control instructions (for direct adjustment of the stimulation configuration) and/or the adaptation instructions (also referred to indirect control instructions, including the adjustment of the closed-loop control algorithm). The RC configuration can be programmed to define commands entered using the RC. In one embodiment, the RC configuration can provide a toggle switch (e.g., a button) for toggling between enabling the adjustment of the closed-loop control algorithm and disabling the adjustment of the closed-loop control algorithm. In one embodiment, the RC configuration can disable the user input device of the RC (e.g., for research study purposes, such as placebo control).

FIG. 12 illustrates an embodiment of portions of a display on a user interface allowing for programming of the RC configuration for interpretation of patient input. The user can select a “TOGGLE” screen, a “SCALE” screen, or a “SET” screen, for example, by respectively clicking on the TOGGLE″, “SCALE”, or “SET” tab. These screens each allow the user to program various aspects of the RC configuration. FIG. 12 shows the TOGGLE screen is selected. Within the TOGGLE screen, the user can enable and disable features related to the direct control (direct adjustment of the stimulation configuration) and adaptation (adjustment of the closed-loop control algorithm). For example, under “How should RC Input be Interpreted?”, the user can select options from the following:
“Direct Control, No Adaptation”: allowing for the direct control without adaptation, to be applied instantly when selected;
“Direct Control, Adaptation”: allowing for the direct control and the adaptation, the direct control to be applied instantly, the adaptation to be applied instantly (if Delay not selected) or after a delay of a specified number of button clicks or a specified time interval (if Delay selected); and
“No Direct Control, Adaptation”: allowing for the adaptation but not the direct control, the adaptation to be applied instantly (if Delay not selected) or after a delay of a specified number of button clicks or a specified time interval (if Delay selected).
   Under “Closed Loop Parameters to Adapt, If any”, the user can select stimulation parameters for which the closed-loop control algorithm is to be adjusted using the RC. Under “User Override”, the user can select the option of allowing the closed-loop control to be overridden using the RC. The override can be forced on when “No Direct Control, Adaptation” is selected. The user can define how the option of override is to be selected (e.g., by holding a button for a specified time interval or clicking for a specified number of times, as illustrated in FIG. 12). The user can also select a type of override from a list of options (e.g., direct control, open-loop stimulation, turning the adaptation off, and reset the adaptation to default).

### Example 2: Setpoint

The RC programming circuit can allow the RC configuration to be programmed to enable the user input device of the RC to receive the patient input for causing gradual shifts in a setpoint and/or a tolerance range around that setpoint. The setpoint can be, for example, a set value for a physiological parameter for comparing with a value of that physiological parameter measured from a sensed signal indicative of a response of the patient to the neurostimulation. The RC programming circuit can determine adjustments of the setpoint and/or the tolerance range around the setpoint based on the patient input (e.g., time, location, and/or content of the patient input) and various associated variables (e.g., parameters indicative of the patient's state and/or conditions). In one embodiment, The RC programming circuit can infer the setpoint and/or the tolerance range over which changes are implemented (or not implemented) in the closed-loop control algorithm using machine learning.

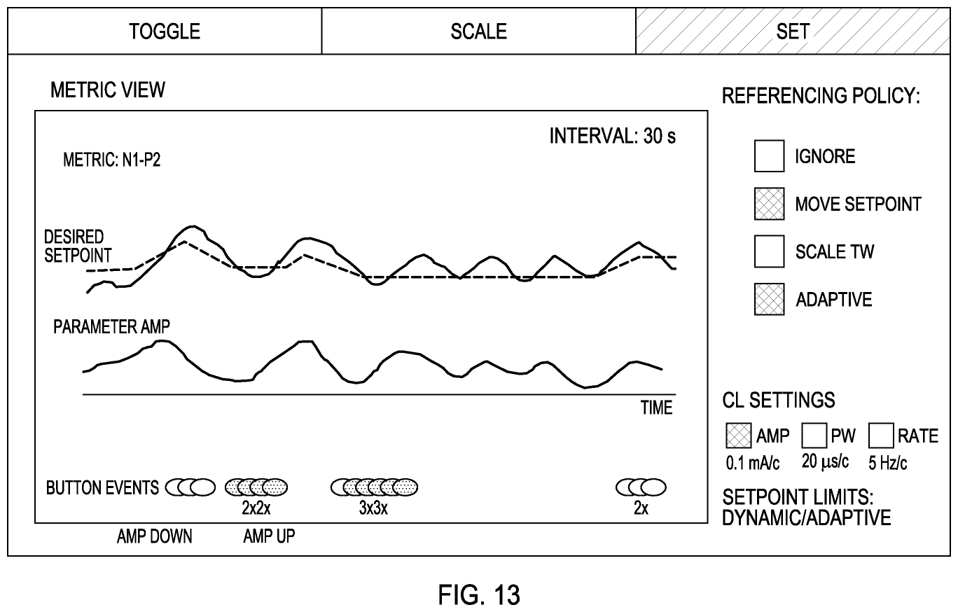

FIG. 13 illustrates an embodiment of portions of a display on a user interface allowing for adjustment of the setpoint. Shown in FIG. 13 is the same portions of the display shown in FIG. 12 but with the SET screen selected. The SET screen presents a Metric View showing one metric as an example, while multiple metrics can be presented simultaneous as needed. The metric can be a parameter measured from neural responses evoked by neurostimulation pulses. In an example, as shown in FIG. 13, the neural responses include evoked compound action potentials (ECAPs) indicative of dorsal column responses (e.g., as seen on an electrospinographic signal). Morphological features of the ECAP (herein referred to as “ECAP features”) that can be used in the present subject matter include, but are not limited to:
N1: the first negative peak in an evoked response that is correlated to the response of faster fibers such as Aβ fibers and myelinated fibers; and
P2: the second positive peak in the evoked response that is correlated with response of slower fibers.
   Various neural response parameters (metrics) can be derived from characteristics of the evoked responses as seen on the neural signal. In various embodiments, the one or more neural response parameters can be measured from the neural signal using the ECAP features. Examples of the one or more neural response parameters generated by detecting and measuring ECAP features for analyzing neural activation and/or controlling delivery of neurostimulation include:
N1-P2 latency: time between N1 and P2;
N1 latency: time interval between start of recording frame (e.g., the neurostimulation pulse) and N1;
P2 latency: time interval between start of recording frame (e.g., the neurostimulation pulse) and P2;
N1-P2 range: N1 to P2 amplitude (the difference between amplitudes of N1 and P2);
Dynamic curve length (CL): curve length measured from the sensed neural signal between N1 and P2; and
Dynamic area under the curve (AUC): the area between the sensed neural signal and a baseline, measured between N1 and P2.
   Shown as an example, the “MERTIC: N1-P2” in FIG. 13 is a curve of the N1-P2 range over time with respect to the setpoint (i.e., the N1-P2 value targeted by the neurostimulation). The “Parameter AMP” in FIG. 13 is a curve of the stimulation amplitude (pulse amplitude) over time.

The Metric View as shown in FIG. 13 presents a metric (N1-P2), a stimulation parameter (AMP, a pulse amplitude), and the setpoint (a set value for N1-P2). The pulse amplitude (AMP) is adjusted to maintain N1-P2 around the setpoint. The SET screen also allows the user to select a “Referencing Policy” for presented options (functions that can be enabled for the RC) including the following:
Ignore: no adjustment to the closed-loop control algorithm;
Move Setpoint: adjustment of the setpoint allowed;
Scale TW: adjustment of therapy window (TW, to be discussed below) allowed; and
Adaptive: adjustment of the closed-loop control algorithm allowed.
   The SET screen further allows the user to select “CL Settings”, i.e., stimulation parameter(s) with respect to which the closed-loop control algorithm is adjustable.

In the example as illustrated in FIG. 13, the RC is used to increase and decrease the pulse amplitude (AMP), for example by using specified types of button events). The setpoint is adjusted automatically (e.g., by the stimulator programming circuit of the RC) when there is concordance between the button events and the closed-loop control algorithm (i.e., when the adjustment made using the RC and the measured performance of the algorithm indicate a need for adjusting the setpoint). The stimulation parameters (e.g., AMP in FIG. 13) may have already been adjusted by the closed-loop control algorithm for maintaining the neurostimulation around the setpoint when the button events occur, and these stimulation parameters are still adjusted to maintain the effectiveness of the neurostimulation around setpoint or both setpoint and a therapeutic window (TW). In an example, lead placement in the patient may be sensitive to motion (e.g., body motion associated with the patient's respiration), and the patient also changes posture, thereby necessitating an adjustment in the neurostimulation to maintain therapeutic effectiveness. Such an adjustment of the neurostimulation, if performed aggressively enough (e.g., using direct control with the RC), can trigger the neurostimulation system to adjust the setpoint (in addition to adjusting one or more stimulation parameters)) for future adjustment of the neurostimulation around the new setpoint.

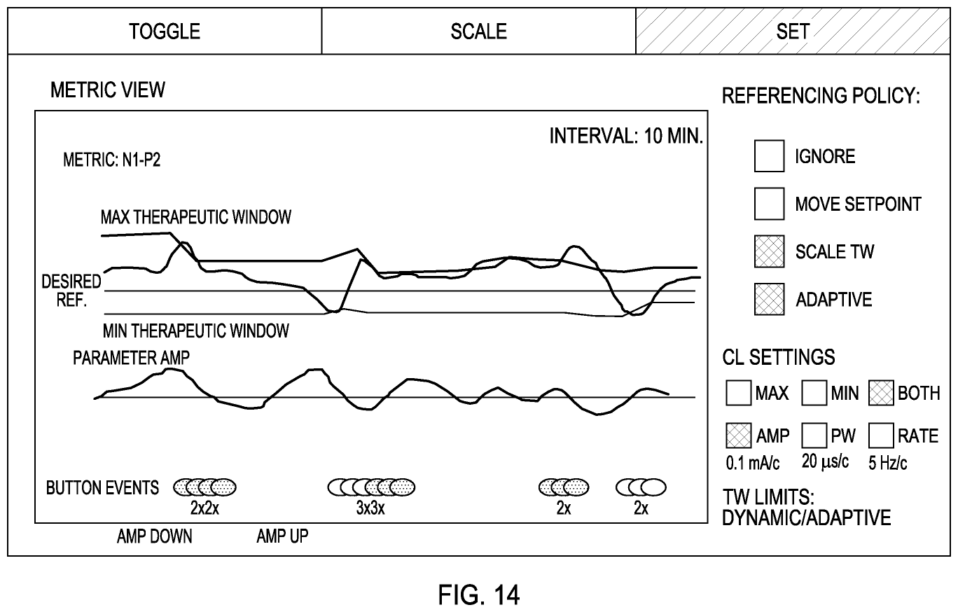

FIG. 14 illustrates an embodiment of portions of a display on a user interface allowing for adjustment of the therapeutic window (TW). Shown in FIG. 14 is the same SET screen shown in FIG. 13 but with the Scale TW selected under the Referencing Policy. TW is a range of intensity of the neurostimulation determined for the patient to receive intended (effective and safe) therapy. TW can be expressed in terms of results of the neurostimulation (e.g., N1-P2 as shown in FIG. 14), i.e., expressed as a range of a metric measured from the patient receiving the neurostimulation.

In the example as illustrated in FIG. 14, the RC is used to increase and decrease the pulse amplitude (e.g., using specified types of button events). The TW is adjusted automatically (e.g., by the stimulator programming circuit of the RC) to a direction that depends on whether there is concordance between the button events and the closed-loop control algorithm (i.e., whether the adjustment made using the RC and the measured performance of the algorithm indicate a need for adjusting the setpoint). The adjustments can include expanding the TW (increasing the distance between maximum and minimum therapeutic thresholds) when there is concordance and narrowing the TW (decreasing the distance between maximum and minimum therapeutic thresholds) when there is discordance between the button events and the closed-loop control algorithm. The TW can be adjusted unidirectionally (changing one of the maximum and minimum therapeutic thresholds) or bidirectionally (changing both of the maximum and minimum therapeutic thresholds). The stimulation parameters (e.g., AMP in FIG. 14) may have already been adjusted by the closed-loop control for maintaining the neurostimulation around the setpoint when the button events occur, and unidirectional change can occur automatically in response to the metric exceeding the TW but patient takes no action after a specified period. In an example, the patient experiences a perturbation that triggers several manual adjustments to therapy (e.g., postural change, goes to/returns from work or athletic activity, household chore, etc. . . . ). The TW is adjusted accordingly with button clicks and is scaled (asymmetrically, here) according to concordance or discordance between clicks and measures. In various embodiments, the TW and the setpoint can be adjusted as a pair. The setpoint is to be set within the TW (i.e., between the lower limit value of the TW and the upper limit value of the TW), so a default position, for example, is to adjust both the TW and the setpoint by the same amount when either the TW or the setpoint is to be adjusted. If the user wishes to modify one of these two parameters and hold the other unchanged, the inequality relationship above can be checked, maintained, and, when considered necessary, used to warn the user about possible conflicts.

### Example 3: Weighting

The RC programming circuit can allow the RC configuration to be programmed to enable the user input device of the RC to receive the patient input as one or more metrics (akin to one or more sensed parameters) under the direct control. The patient input (representing the one or more metrics) is used as an input for changing one or more stimulation parameters under the direct control, using a relationship between the one or more stimulation parameters and the patient input. For example, when “Direct” (with or without “Adaptive”) is selected under Manual Input Policy, button events can be used as a metric itself (akin to posture, breathing, N1-P2 amplitude, or the like) and control the pulse amplitude (AMP) according to the button events (e.g., number and direction of clicks), in place of or in addition to using button events to control AMP directly. The button events do not necessarily control the stimulation parameters according to a 1:1 mapping between a change in the stimulation parameters and a certain button event. A button event can be programmed to directly result in a change of a specified primary stimulation parameter (e.g., AMP) or to indirectly result in one or more other stimulation parameters (e.g., pulse width, pulse frequency, and the like) to be changed according to their relationship with the primary stimulation parameter under specified programming rules.

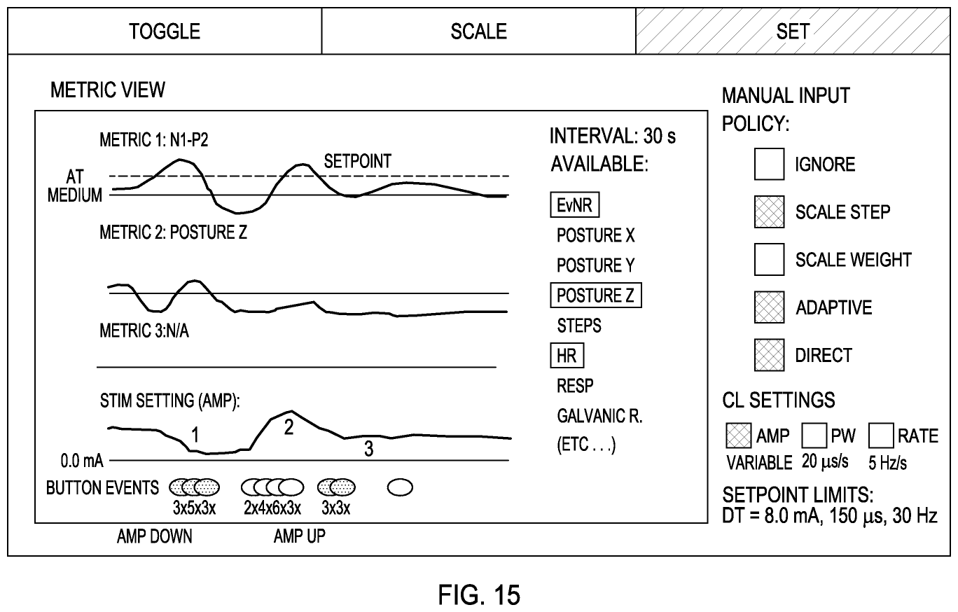

FIG. 15 illustrates an embodiment of portions of a display on a user interface allowing for use of patient input as the one or more metrics. Shown in FIG. 15 is the same portions of the display shown in FIG. 12 but with the SET screen selected. Examples of metrics that can be entered by button events, as shown in FIG. 15, include:
EvNR; evoked neural response (e.g., N1-P2 and/or one or more other neural response parameters measured from ECAPs as discussed above);
Posture X: a body posture (e.g., lying down);
Posture Y: another body posture (e.g., sitting);
Posture Z: another body posture (e.g., standing);
Steps: an indicator of movement of the patient (e.g., a pedometer reading);
HR: heart rate;
Resp: respiration rate; and
Galvanic R.: galvanic skin response.
   The user can select functions related to using the patient input as one or more metrics to be enabled for the RC under Manual Input Policy. Examples of such functions, as shown in FIG. 15, include:
Ignore: no patient input is to be taken as a metric;
Scale Step: scaling relationship between the patient input and the metric (e.g., increase or decrease step size of the metric corresponding to each button click);
Scale Weight: scaling weight for each metric for use in controlling the stimulation parameter(s);
Adaptive: the closed-loop control algorithm is adjustable;
Direct: the patient input (e.g., the button events) directly adjusts one or more stimulation parameters (e.g., AMP);
   In a scenario for example, the patient changes posture (e.g., lies down), gets “zinged”, and interacts with the RC several times because the closed-loop control algorithm does not adapt quickly enough. The closed-loop control algorithm can be adjusted to change the step size of the closed-loop control based on the button events (e.g., frequency, duration, and/or rate of button clicks), with or without directly adjusting the stimulation parameters. Examples of such adjustments, as illustrated in FIG. 15 with the numbers 1-3 labeled, include:
1. step size increases if button click direction is in the same direction to which the closed-loop control algorithm progresses, if no button click has occurred in a specified period;
2. step size decreases if button click direction is in opposite direction to which the pulse amplitude (AMP) progresses and/or if button clicks in the opposite direction has happened within a specified (short) period; and
3. step size increments can be held constant (or ignored) for isolated button events.
   Direction of change in the closed-loop control can act, for example, on a hyperbolic function resembling a “spike-timing dependent plasticity” curve, or a sigmoid function.

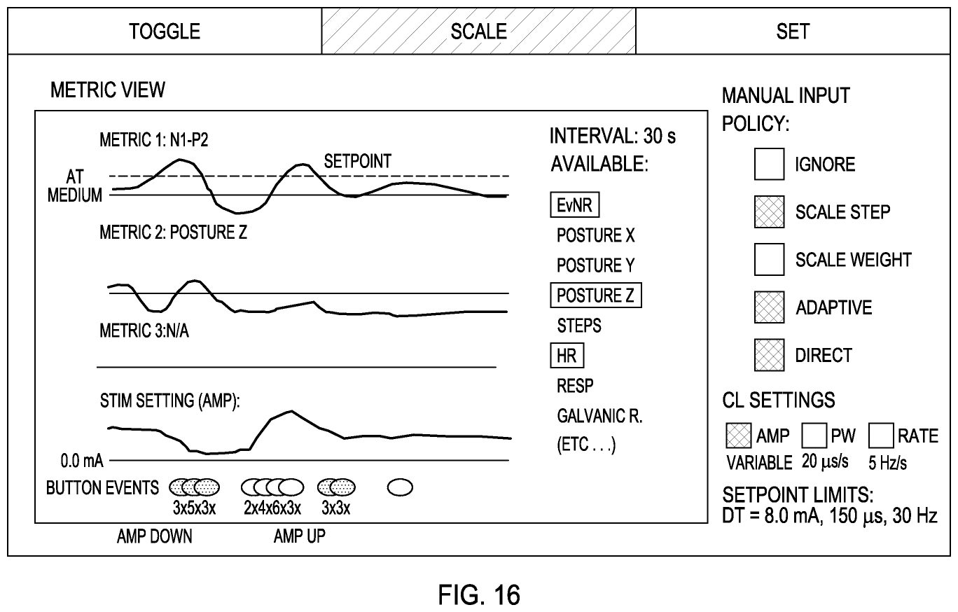

FIG. 16 illustrates an embodiment of portions of a display on a user interface allowing for use of patient input as the one or more metrics with step scaling. Shown in FIG. 16 is the same portions of the display shown in FIG. 15 but with the SCALE screen selected. FIG. 16 illustrates an example in which “Scale Step” under Manual Input Policy is selected and “Variable” control under CL Settings is selected. The “Variable” control when selected can enable configuration of step size default, step size increment, step size settling time to default or another value (e.g., decrease step size to baseline over a specified period), etc. The “Adaptive” option under Manual Input Policy when selected can additionally allow configuration of settings such as tying rate to multiple variables (e.g., increase/decrease with Posture Z), using machine learning among one or other variables (e.g., N1-P2 change trends) to adjust step size in changing the closed-loop control, etc.

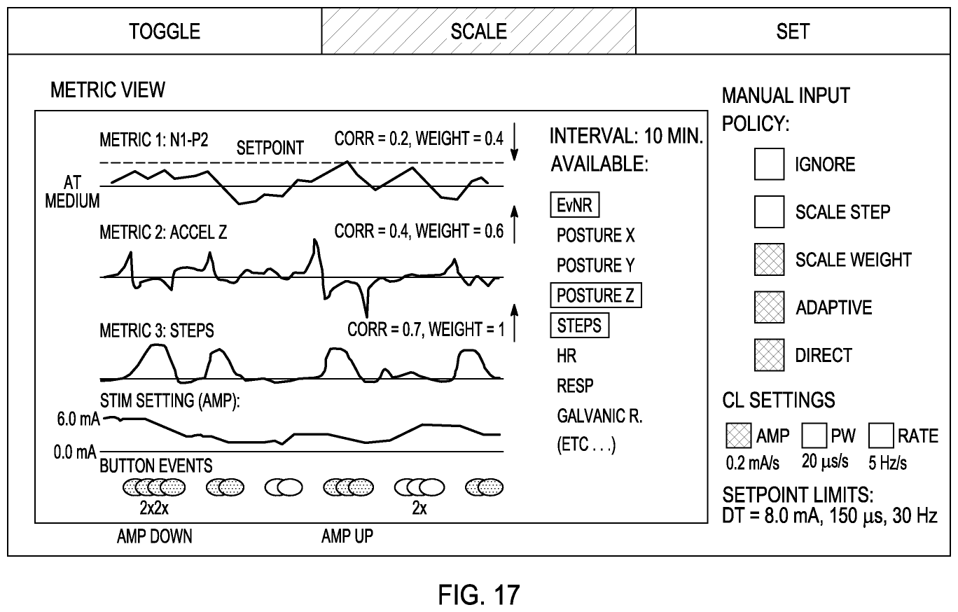

FIG. 17 illustrates an embodiment of portions of a display on a user interface allowing for use of patient input as the one or more metrics with weight scaling. Shown in FIG. 17 is the same portions of the display shown in FIG. 15 but with the SCALE screen selected. FIG. 17 illustrates an example in which “Scale Weight” under Manual Input Policy is selected and the Metric View presents N1-P2, Posture Z and Steps. In this example, the patient hypothetically adjusts the neurostimulation beyond internal sensing in a manner that is more correlated with an external measure (e.g., standing posture or steps taken). The neurostimulation system automatically analyzes this scenario (e.g., through correlation vs. fixed time interval) and to result in de-weighting of N1-P2 in controlling the neurostimulation and re-weighting towards Posture Z and/or Steps. The setpoint and the setting steps of the closed-loop control algorithm do not change as shown in FIG. 17, but either or both can change concurrently with the weight scaling.

Some metrics may be “riskier” or “off limits” due to their inherent ability to destabilize the closed loop control algorithm, their importance in controlling the therapy (relative to other parameters), and/or the sensitivity of other stimulation settings to that parameter. Weighting as applied to such metrics can be limited or qualified. Examples of limiting or qualifying in weighting include:
setting an absolute or relative limit on a step size; and
including or excluding one or more specific parameters based on calibration, a priori data, and/or real-time or substantially real-time adjustments.

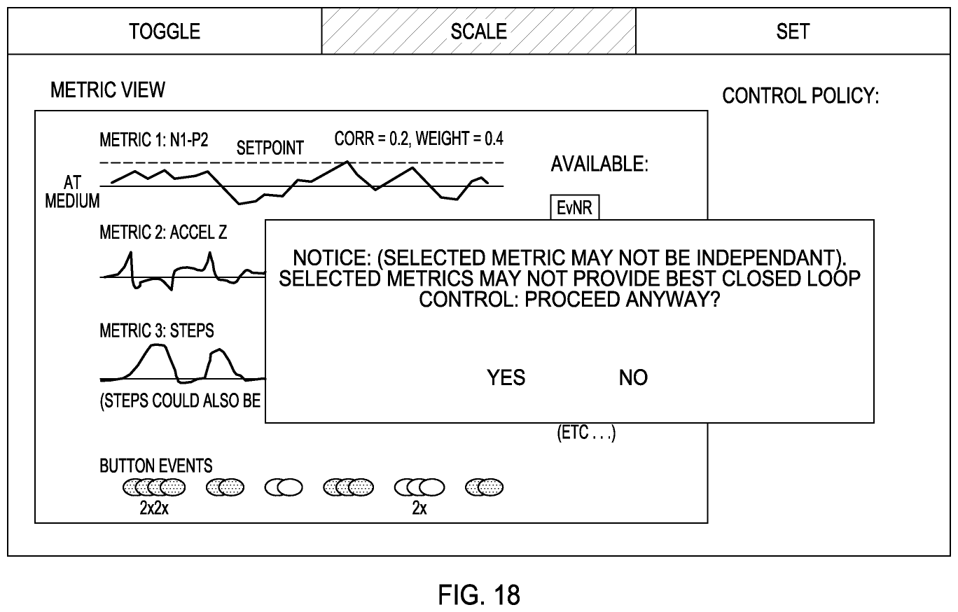

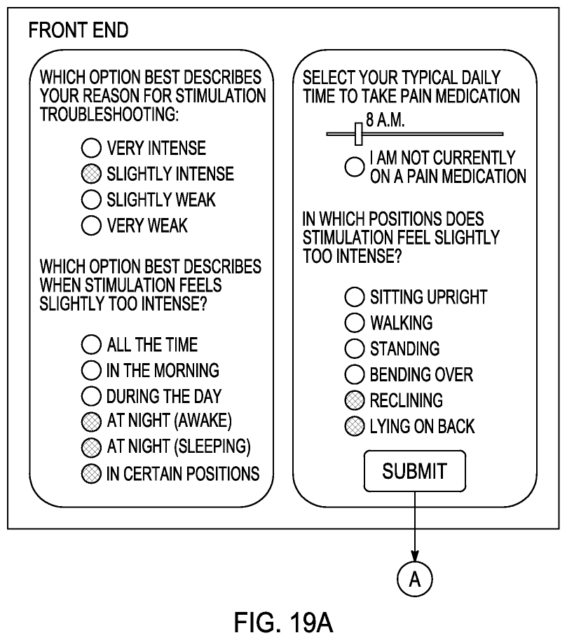

FIG. 18 illustrates an embodiment of portions of a display on a user interface showing a warning message on accepting the patient input. Shown in FIG. 18 is the same portions of the display shown in FIGS. 16 and 17. When parameters (metrics) are known to covary, the RC programming circuit can cause a warning message to be displayed, as illustrated in FIG. 18, to inform the user that the variables selected may not produce optimal outcomes due to, for example, collinearity or autocorrelation. The user can be given an option to select between proceeding or re-selecting parameters.

### Example 4: Survey

The RC programming circuit can allow the RC configuration to be programmed to enable the user input device of the RC to receive the patient input as response to a survey. The survey can include, for example, a questionnaire for the patient, as part of a diagnosis to identify needs of the patient that can potentially be met by the neurostimulation. Results of the survey can be used to adjust the closed-loop control algorithm. For example, the patient can be asked a series of questions for which the answers can relate the patient's conditions and needs to neurostimulation therapy settings. The patient can clarify a source of dissatisfaction with the neurostimulation (e.g., “briefly too intense/weak” or “always too intense/weak”) as well as its relation to posture/actions (“when sleeping, “when bending over”, etc.). Responses to the survey can be routed through a decision tree leading to the neurostimulation therapy settings. When applicable, the responses to the survey can be used to adjust the closed-loop control algorithm (e.g., default step sizes, toggling between TW and step, metrics to use, etc.).

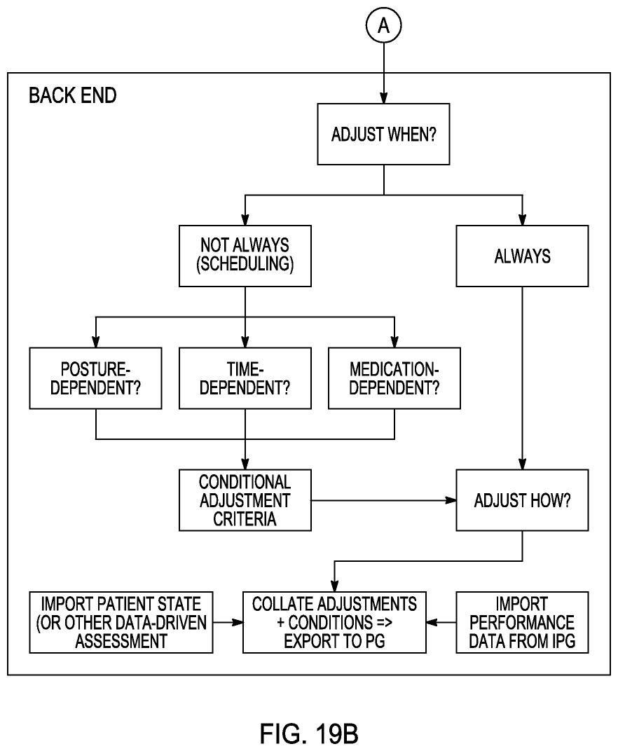

FIGS. 19A-19B illustrate an embodiment of portions of a display on a user interface (FIG. 19A) and a decision tree (FIG. 19B) used in survey-based adjustments of the closed-loop control of neurostimulation. The “Front End”, as shown in FIG. 19A, can represent a screen of the user interface of the RC that is used present the survey questions to the patient. After the patient completes the survey and clicks “Submit”, the patient's responses to the survey are sent to the “Back End”, as shown in FIG. 19B, which can represent processing circuitry of the RC (e.g., the stimulator programming circuit). The processing circuitry processes the patient's responses using the decision tree as illustrated in FIG. 19B. Examples of adjustments using the decision tree include:
scheduling—to adjust the closed-loop control only when specified conditions are met, for example:
   Posture-dependent—an accelerometer in the implantable stimulator can be used to indicate the patient's posture or posture changes, and the closed-loop control is to be adjusted only in response to a detection of a specified type of posture or posture change;
   Time-dependent—a clock in the implantable stimulator can be used to indicate time of day, and the closed-loop control is to be adjusted only during daily times when pain and/or sensitivity of the patient is known to be particularly severe; and
   Medication-dependent—adjustment of the closed-loop control can be triggered by a change in the medication taken by the patient or a change in the effectiveness of the medication (e.g., with the RC configured to track the administration and effectiveness of the medication); and
adjusting, for example:
   Setpoint change—the amount of adjustment of the setpoint for the closed-loop control can be dependent on the amount of discomfort perceived by the patient (e.g., raised/lowered 10% for “slightly weak/intense” and 25% for “very weak/intense”);
   Threshold change—the amount of adjustment of the TW for the closed-loop control can be dependent on the amount of discomfort perceived by the patient (e.g., TW narrowed for both “weak” and “intense”, upper threshold lowered for “intense”, lower threshold raised for “weak”;
      In an example of medication-dependent adjustment of the closed-loop control, if timing of reported medication-taking and timing/direction of troubleshooting suggests that the effectiveness of the medication is wearing off. For example, if timing of troubleshooting minus timing of medication-taking is 4 hours or more, and the direction of troubleshooting is towards “too weak”, then medication dependency can be a primary factor for the adjustment of the closed-loop control. The RC can be programmed to include a medication-tracking application that can trigger adjustment of the closed-loop control algorithm based on changes in medication and/or changes in effectiveness of the medication.

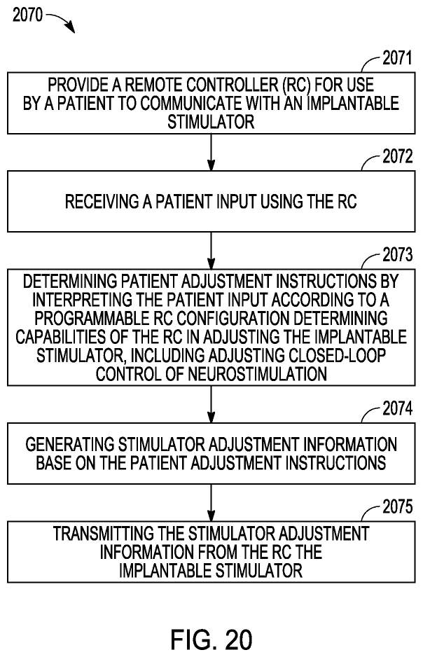

FIG. 20 illustrates an embodiment of a method 2070 for adjusting closed-loop control of neurostimulation based on patient input. In one embodiment, method 2070 is performed using system 900. In one embodiment, a non-transitory computer-readable storage medium includes instructions, which when executed by a system, such as system 900, cause the system to perform method 2070. Examples of the remote controller (RC) used in method 2070 include RC 932 and 1032. Method 2070 as discussed below is part of a method for delivering neurostimulation to a patient and controlling the neurostimulation by a user. The neurostimulation is delivered from an implantable stimulator (e.g., implantable stimulator 902) that can control the delivery of the neurostimulation according to a stimulation configuration and adjust the stimulation configuration by using a closed-loop control algorithm.

At 2071, an RC configured to be used by the patient is provided. The RC can wirelessly communicate with the implantable stimulator. The RC is given to the patient for adjusting the delivery of the neurostimulation by the patient or another person on behalf of the patient.

At 2072, a patient input is received using the RC. At 2073, patient adjustment instructions are determined by interpreting the patient input according to an RC configuration. The RC configuration is programmable for modifying capabilities of the RC in adjusting the stimulation configuration and adjusting the closed-loop control algorithm. In one embodiment, the RC configuration is programmable using the RC itself, such as by an authorized user. For example, an RC user input can be received using a user interface of the RC with a password-protected access to the programming of the RC configuration, and the RC configuration is adjusted using the RC user input. In one embodiment, the RC configuration is programmable using another device accessible by the authorized user. The RC can receive RC programming information using its telemetry circuit, and the RC configuration is adjusted using the RC programming information. An example of the other device accessible by the authorized user is a clinician's programmer (CP), such as CP 930 or CP 1130, that is configured for use by the user. A CP user input is received using a user interface of the CP. The RC programming information is generated based on the CP user input and transmitted to the RC. Various examples of adjustment of the RC configuration include:
adjusting the RC configuration to enable the RC to receive the patient input for at least one of adjusting the stimulation configuration or adjusting the closed-loop control algorithm;
adjusting the RC configuration to enable the RC to receive the patient input for causing gradual shifts in at least one of a setpoint or a tolerance range around that setpoint (the setpoint is a parameter of the closed-loop control algorithm associated with a measurable result of the delivery of the neurostimulation);
adjusting the RC configuration to enable the RC to receive the patient input as one or more metrics that are directly to adjust the stimulation configuration directly; and
adjusting the RC configuration to enable the RC to receive the patient input as a response to a survey, where the response is used to adjust at least one of the stimulation configuration or the closed-loop control algorithm based on the response to the survey.

At 2074, stimulator adjustment information is generated based on the patient adjustment instructions. The stimulator adjustment information can include direct control adjustment information for adjusting the stimulation configuration directly and/or adaptation adjustment information for adjusting the closed-loop control algorithm. The usage of the RC in adjusting the delivery of the neurostimulation from the implantable stimulator can be tracked by the RC. In one embodiment, the usage of the RC in generating the direct control adjustment information and the usage of the RC in generating the adaptation adjustment information are individually tracked to provide a record on how the RC functions are utilized by the patient.

At 2075, the stimulator adjustment information is transmitted from the RC to the implantable stimulator. This causes the delivery of the neurostimulation from the implantable stimulator to be controlled according to the adjusted stimulation configuration and/or the adjusted closed-loop control algorithm.

In various embodiments, the present subject matter can be applied to existing neurostimulation systems with closed-loop control. For example, an RC and/or CP may be configured for programming the RC configuration, while it may not be necessary to modify an existing implantable stimulation that is already configured to include closed-loop control of neurostimulation.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation to a patient and controlling the neurostimulation by a user, the neurostimulation delivered from an implantable stimulator configured to control the delivery of the neurostimulation according to a stimulation configuration and to adjust the stimulation configuration by using a closed-loop control algorithm, the system comprising:
a remote controller (RC) configured for use by the patient, the RC comprising:
an RC telemetry circuit configured to provide wireless communication between the RC and the implantable stimulator and to transmit stimulator adjustment information to the implantable stimulator, the stimulator adjustment information including direct control adjustment information for adjusting the stimulation configuration and adaptation adjustment information for adjusting the closed-loop control algorithm;
an RC programming control circuit configured to generate the stimulator adjustment information based on patient adjustment instructions; and
an RC user interface including an RC user input device configured to receive a patient input and a stimulator programming circuit configured to determine the patient adjustment instructions by interpreting the patient input according to an RC configuration that is programmable for modifying capabilities of the RC in adjusting the stimulation configuration and adjusting the closed-loop control algorithm.

2. The system of claim 1, wherein the stimulator programming circuit is configured to enable and disable each function of RC functions using the user input according to the RC configuration, the RC functions allowing the delivery of the neurostimulation from the implantable stimulator to be adjusted using the RC and including one or more functions each allowing one or more aspects of the closed-loop control algorithm to be adjusted using the RC.

3. The system of claim 1, further comprising an RC programming circuit configured to receive RC programming information and to determine the RC configuration based on the RC programming information for enabling the RC user input device to receive the patient input for at least one of adjusting the stimulation configuration or adjusting the closed-loop control algorithm.

4. The system of claim 3, wherein the RC user interface comprises the RC programming circuit, and the RC user input device is configured to receive the RC programming information.

5. The system of claim 3, wherein the RC is configured to receive the RC programming information using the RC telemetry circuit and to program the RC configuration using the received RC programming information.

6. The system of claim 3, wherein the RC programming circuit is configured to allow the RC configuration to be defined to allow the RC user input device to receive the patient input for adjusting the stimulation configuration while overriding the closed-loop control algorithm.

7. The system of claim 3, wherein the RC programming circuit is configured to allow the RC configuration to be defined to enable the RC user input device to receive the patient input for causing gradual shifts in at least one of a setpoint or a tolerance range around that setpoint, the setpoint being a parameter of the closed-loop control algorithm associated with a measurable result of the delivery of the neurostimulation.

8. The system of claim 3, wherein the RC programming circuit is configured to allow the RC configuration to be defined to enable the RC user input device to receive the patient input as one or more metrics, and the stimulator programming circuit is configured to adjust the stimulation configuration directly using the one or more metrics.

9. The system of claim 8, wherein the RC programming circuit is configured to allow the RC configuration to be defined to enable the RC user input device to receive at least one of a weighting factor or a scaling factor for each parameter of the one or more metrics, and the stimulator programming circuit is configured to adjust the stimulation configuration directly using the each parameter and the respective at least one of the weighting factor or the scaling factor.

10. The system of claim 3, wherein the RC programming circuit is configured to allow the RC configuration to be defined to enable the RC user input device to receive the patient input as a response to a survey, and the stimulator programming circuit is configured to adjust at least one of the stimulation configuration or the closed-loop control algorithm based on the response to the survey.

11. A method for delivering neurostimulation to a patient and controlling the neurostimulation by a user, the neurostimulation delivered from an implantable stimulator configured to control the delivery of the neurostimulation according to a stimulation configuration and to adjust the stimulation configuration by using a closed-loop control algorithm, the method comprising:
providing a remote controller (RC) configured to be used by the patient and to wirelessly communicate with the implantable stimulator;
receiving a patient input using the RC;
determining patient adjustment instructions by interpreting the patient input according to an RC configuration that is programmable for modifying capabilities of the RC in adjusting the stimulation configuration and adjusting the closed-loop control algorithm;
generating stimulator adjustment information based on the patient adjustment instructions, the stimulator adjustment information including direct control adjustment information for adjusting the stimulation configuration directly and adaptation adjustment information for adjusting the closed-loop control algorithm; and
transmitting the stimulator adjustment information from the RC to the implantable stimulator.

12. The method of claim 11, further comprising tracking a usage of the RC in generating the direct control adjustment information and a usage of the RC in generating the adaptation adjustment information.

13. The method of claim 11, further comprising:
receiving RC user input using a user interface of the RC; and
adjusting the RC configuration using the RC user input.

14. The method of claim 11, further comprising:
receiving RC programming information using a telemetry circuit of the RC; and
adjusting the RC configuration using the RC programming information.

15. The method of claim 14, further comprising:
providing a clinician's programmer (CP) configured for use by the user;
receiving a CP user input using a user interface of the CP; and
generating the RC programming information based on the CP user input.

16. The method of claim 11, further comprising adjusting the RC configuration to enable the RC to receive the patient input for at least one of adjusting the stimulation configuration or adjusting the closed-loop control algorithm.

17. The method of claim 11, further comprising adjusting the RC configuration to enable the RC to receive the patient input for causing gradual shifts in at least one of a setpoint or a tolerance range around that setpoint, the setpoint being a parameter of the closed-loop control algorithm associated with a measurable result of the delivery of the neurostimulation.

18. The method of claim 11, further comprising adjusting the RC configuration to enable the RC to receive the patient input as one or more metrics that are used to directly adjust the stimulation configuration.

19. The method of claim 11, further comprising adjusting the RC configuration to enable the RC to receive the patient input as a response to a survey, the response used to adjust at least one of the stimulation configuration or the closed-loop control algorithm.

20. A non-transitory computer-readable storage medium including instructions, which when executed by a system, cause the system to perform a method for delivering neurostimulation to a patient and controlling the neurostimulation by a user, the neurostimulation delivered from an implantable stimulator configured to control the delivery of the neurostimulation according to a stimulation configuration and to adjust the stimulation configuration by using a closed-loop control algorithm, the method comprising:
communicating with the implantable stimulator wirelessly using a remote controller (RC) configured for use by the patient;
receiving a patient input using the RC;
determining patient adjustment instructions by interpreting the patient input according to an RC configuration that is programmable for modifying capabilities of the RC in adjusting the stimulation configuration and adjusting the closed-loop control algorithm;
generating stimulator adjustment information based on the patient adjustment instructions, the stimulator adjustment information including direct control adjustment information for adjusting the stimulation configuration directly and adaptation adjustment information for adjusting the closed-loop control algorithm; and
transmitting the stimulator adjustment information from the RC to the implantable stimulator.
